(19) United States
(12) Patent Application Publication
(10) Pub. No.: US 2010/0256085 A1
(43) Pub. Date: Oct. 7, 2010

(54) Toll-Like Receptor Agonist Regulation of VEGF-Induced Tissue Responses

(75) Inventor: Jack Elias, New Haven, CT (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Yale University, New Haven, CT (US)

(21) Appl. No.: 12/445,692

(22) PCT Filed: Oct. 16, 2007
(86) PCT No.: PCT/US07/22009

§ 371 Date: Jun. 15, 2010

Related U.S. Application Data
(60) Provisional application No. 60/852,309, filed on Oct. 16, 2006.

(51) Int. Cl. A61K 31/708; A61K 31/522; A61K 31/715; A61K 31/437; A61P 11/00; A61P 35/00; A61P 31/00; A61P 29/00; A61P 25/24; A61P 25/22; A61P 21/02

(52) U.S. Cl. 514/47; 514/263.31; 514/54; 514/293

(57) ABSTRACT

The present invention encompasses compositions and compounds as well as methods of their use for the regulation of a VEGF-induced tissue response. A VEGF-induced tissue response may include angiogenesis, inflammation, increased vascular permeability, increased vascular leak, hemorrhage, or mucus metaplasia. As such, the present invention encompasses methods of treating diseases where a VEGF-induced tissue response is part of the disease's clinical presentation. Specifically, the present invention provides compounds and compositions as well as methods for treating acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer.

### BACKGROUND OF THE INVENTION

Vascular endothelial cell growth factor (VEGF) is a critical regulator of the angiogenesis that occurs during physiologic responses such as reproduction and development and pathologic responses as diverse as those in tumor neovascularization, obesity, retinopathies, coronary artery disease and ischemic vascular disorders (Tammela et al., 2005, Cardiovasc. Res. 65:550-63; Ferrara et al., 2003, Nature Medicine 9:669-76; Reynolds et al., 2001, Biol. Reprod. 64:1033-40; Silha et al., 2005, Int. J. Obes. 29:1308-14; Khurana et al., 2005, Circulation 112:1813-24 ; Wilkinson-Berka, 2004, Curr. Pharm. Des. 10:3331-48). Studies of these responses have demonstrated that VEGF induces the proliferation, sprouting, migration and tube formation of endothelial cells, regulates endothelial cell survival, induces vasodilatation, and regulates vascular permeability (Tammela et al., 2005, Cardiovasc. Res. 65:550-63; Ferrara et al., 2003, Nature Medicine 9:669-76). Recent studies, however, have demonstrated that, VEGF also has prominent inflammatory, immune and remodeling effects on nonvascular tissues (Lee et al., 2004, Nature Medicine 10:1095-103; He et al., J. Clin. Invest. 115:1039-48). The mechanism by which VEGF exerts its endothelial cell effects has been the topic of intense investigation. However, the processes that regulate these responses at sites of pathology and the mechanisms of these regulatory events have not been defined. In addition, very little is known about the mechanisms that VEGF uses to induce extra-vascular responses in the lung or other tissues and the processes that regulate these responses are also poorly understood.

Exaggerated Th2 inflammation and airway remodeling are cornerstones in the pathogenesis of asthma (Elias et al., 2003, J. Clin. Invest. 111: 291-7; Elias et al., 1999, J. Clin. Invest. 104:1001-6; Wills-Karp et al., 2003, Curr. Opin. Pulm. Med. 9:21-7). Increases in vessel number, vessel size, vessel surface area and vascular leak are prominent features of these remodeling responses (Charan et al., 1997, Eur. Respir. J. 10:1173-80; Hogg, 1999, Thorax 54:283; Hoshino et al., 2001, J. Allergy Clin. Immunol. 107:1034-8; Hoshino et al., 2001, J. Allergy Clin. Immunol. 107:295-301; Lee et al., 2001, J. Allergy Clin. Immunol. 107:1106; Li et al., 1997, Am. J. Respir. Crit. Care Med. 156: 229-33; Orsida et al., 2001, Am. J. Respir. Crit. Care Med. 164:117-21; Salvato et al., 2001, Thorax 56: 902-6; Vrugt et al., 2000, Eur. Respir. J. 15:1014-21). In keeping with these vascular findings, exaggerated levels of VEGF have been detected in tissues and biologic samples from patients with asthma (Lee et al., 2004, Nature Medicine 10:1095-103; Hoshino et al., 2001, J. Allergy Clin. Immunol. 107:1034-8; Hoshino et al., 2001, J. Allergy Clin. Immunol. 107:295-301; Asai et al., 2002, J. Allergy Clin. Immunol. 110:571-5; Kanazawa et al., 2002, Thorax 57:885-8). In these patients VEGF levels correlate directly with disease activity (Lee et al., 2001, J. Allergy Clin. Immunol. 107:1106) and inversely with airway caliber and airway hyperresponsiveness (AHR) (Hoshino et al., 2001, J. Allergy Clin. Immunol. 107:1034-8; Hoshino et al., 2001, J. Allergy Clin. Immunol. 107:295-301; Asai et al., 2002, J. Allergy Clin. Immunol. 110:571-5; Kanazawa et al., 2002, Thorax 57:885-8). VEGF was originally postulated to contribute to asthma via its effects on vascular permeability (Charan et al., 1997, Eur. Respir. J. 10:1173-80; Antony et al., 2002, J. Allergy Clin. Immunol. 110:589-95; Thurston et al., 2000, Nature Medicine 6:460-3). Recent results have refined this concept by demonstrating that (a) the transgenic overexpression of VEGF in the murine lung induces an asthma-like phenotype with inflammation, parenchymal and vascular remodeling, tissue edema, mucus metaplasia, myocyte hyperplasia, airway hyperresponsiveness (AHR), dendritic cell (DC) hyperplasia and activation, enhanced respiratory antigen sensitization and augmented Th2 inflammation (Lee et al., 2004, Nature Medicine 10:1095-103); (b) VEGF is required for antigen-induced Th2 inflammation and IL-4 and IL-13 elaboration (Lee et al., 2004, Nature Medicine 10:1095-103); and, (c) nitric oxide synthase (NOS; eNOS and iNOS) production of nitric oxide (NO) plays critical roles in the generation of many of these VEGF tissue responses (Bhandari et al., 2006, Proc. Natl. Acad. Sci. U. S. A. 103:11021-6).

The mechanisms of VEGF-induced vascular and extra-vascular pulmonary alterations, however, have not been adequately defined. In addition, the regulation of these VEGF responses at sites of pathology has not been adequately investigated.

Dysregulated VEGF production and inflammation frequently co-exist at sites of pathology. VEGF-induced tissue response such as angiogenesis, inflammation, and remodeling can lead to prominent clinical features associated with numerous diseases and disorders including acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. There is a long-felt need in the art for a therapeutic method of regulating pathological VEGF-induced tissue response. The present invention fills this need.

### SUMMARY OF THE INVENTION

One embodiment of the invention comprises a method of regulating a vascular endothelial growth factor (VEGF)-induced tissue response in a mammal, said method comprising administering to said mammal a therapeutically effective amount of at least one toll-like receptor (TLR) agonist, and further wherein when said TLR agonist is administered to said mammal, said VEGF-induced tissue response is regulated in said mammal. In one aspect of the invention the mammal is a human. In another aspect of the invention the VEGF-induced tissue response comprises increased angiogenesis, tissue inflammation, vascular permeability, vascular leak, hemorrhage, or mucus metaplasia. In another aspect of the invention, the mammal has been diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In still another aspect of the invention, the TLR agonist is administered in combination with at least one other therapeutic agent. In another aspect of the invention, the TLR agonist is administered before, during, or after said therapeutic agent, or a combination thereof. In another aspect of the invention, the therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof. In still another aspect of the invention, aTLR is administered in combination with other treatment modalities, such as chemotherapy, cryotherapy, hyperthermia, radiation therapy, or a combination thereof. In yet another aspect of the invention, the TLR agonist specifically binds to a TLR3, a TLR7, a TLR9, a TLR4, or a combination thereof. In still another aspect of the invention, the TLR agonist comprises a poly(I:C), Gardiquimod, a CpG, a LPS, or a combination thereof.

Another embodiment of the invention comprises a method of treating a vascular endothelial growth factor (VEGF)-induced tissue response in a mammal, said method comprising administering to said mammal a therapeutically effective amount of at least one toll-like receptor (TLR) agonist, and further wherein when said. TLR agonist is administered to said mammal, said VEGF-induced tissue response is attenuated in said mammal. In one aspect of the invention, the mammal is a human. In another aspect of the invention, the VEGF-induced tissue response comprises increased angiogenesis, tissue inflammation, vascular permeability, vascular leak, hemorrhage, or mucus metaplasia. In still another aspect of the invention, the mammal has been diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In yet another aspect of the invention, the TLR agonist is administered in combination with at least one other therapeutic agent. In another aspect of the invention, the TLR agonist is administered before, during, or after said therapeutic agent, or a combination thereof. In another aspect of the invention, the therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof. In another aspect of the invention, a TLR is administered in combination with other treatment modalities, such as chemotherapy, cryotherapy, hyperthermia, radiation therapy, or a combination thereof. In still another aspect of the invention, the TLR agonist specifically binds to a TLR3, a TLR7, a TLR9, a TLR4, or a combination thereof. In still another aspect of the invention, the TLR agonist comprises a poly(I:C), Gardiquimod, a CpG, a LPS, or a combination thereof.

Yet another embodiment of the invention comprises a method of preventing a vascular endothelial growth factor (VEGF)-induced tissue response in a mammal, said method comprising administering to said mammal a therapeutically effective amount of at least one toll-like receptor (TLR) agonist, and further wherein when said TLR agonist is administered to said mammal, said VEGF-induced tissue response is prevented in said mammal. In one aspect of the invention, the mammal is a human. In another aspect of the invention, the VEGF-induced tissue response comprises increased angiogenesis, tissue inflammation, vascular permeability, vascular leak, hemorrhage, or mucus metaplasia. In another aspect of the invention, the mammal is at risk of developing at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In still another aspect of the invention, the TLR agonist is administered in combination with at least one other therapeutic agent. In another aspect of the invention, the TLR agonist is administered before, during, or after said therapeutic agent, or a combination thereof. In another aspect of the invention, the therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof. In yet another aspect of the invention, a TLR is administered in combination with other treatment modalities, such as chemotherapy, cryotherapy, hyperthermia, radiation therapy, or a combination thereof. In another aspect of the invention, the TLR agonist specifically binds to a TLR3, a TLR7, a TLR9, a TLR4, or a combination thereof. In another aspect of the invention, the TLR agonist comprises a poly(I:C), Gardiquimod, a CpG, a LPS, or any combination thereof.

Still another embodiment of the invention comprises a method of treating a mammal diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer, said method comprising administering to said mammal a therapeutically effective amount of at least one toll-like receptor (TLR) agonist. In one aspect of the invention, the TLR agonist is administered in combination with at least one other therapeutic agent. In another aspect of the invention, the TLR agonist is administered before, during, or after the therapeutic agent, or a combination thereof. In another aspect of the invention, the therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof. In another aspect of the invention, a TLR is administered in combination with other treatment modalities, such as chemotherapy, cryotherapy, hyperthermia, radiation therapy, or a combination thereof. In yet another aspect of the invention, the TLR agonist specifically binds to a TLR3, a TLR7, a TLR9, a TLR4, or a combination thereof. In another aspect of the invention, the TLR agonist comprises a poly(I:C), Gardiquimod, a CpG, a LPS, or a combination thereof. In yet another aspect of the invention, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of images depicting the effect of Poly(I:C) on VEGF-induced angiogenesis. FIG. 1A is a photomicrograph depicting tissue where WGF expression was either induced by Dox administration (VEGF+) or in animals administered a control (VEGF−). In addition, animals were either administered Poly(I:C) or a PBS control 1 day prior to the start of Dox administration. Tissue is stained for CD31. Poly(I:C). FIG. 1B is a bar graph depicting the morphometric quantification of the percentage of the surface area of the airway that was covered by blood vessels.

FIG. 2, comprising FIG. 2A and FIG. 2B, is a series of graphs depicting the effect of Poly(I:C) treatment on bronchoalveolar lavage (BAL) total protein and Evans Blue leakage in the lung. FIG. 2A is a graph depicting lower levels of protein in BAL fluids obtained from Tg+ mice treated with Poly (I:C) when compared with Tg+ mice treated with vehicle. FIG. 2B is a graph depicting that there are lower levels of Evan's blue dye leakage in lungs from Tg+ mice treated with poly(LC) compared to Tg+ mice treated with control vehicle.

FIG. 3, is a photograph, depicting BAL fluid obtained from VEGF+ or VEGF− mice treated with either phosphate buffered saline (PBS) or different concentrations of Poly(I:C).

FIG. 4, comprising FIG. 4A through 4E, is a series of graphs depicting inflammatory cell recovery during BAL. FIG. 4A is a graph depicting the total number of inflammatory cells recovered during BAL in either VEGF+ or VEGF− mice who were either treated with vehicle control or Poly (I:C). FIG. 4B is a graph depicting the number of macrophages recovered during BAL in either VEGF+ or VEGF− mice who were either treated with vehicle control or Poly (I:C). FIG. 4C is a graph depicting the number of eosinophils recovered during BAL in either VEGF+ or VEGF− mice who were either treated with vehicle control or Poly (I:C). FIG. 4D is a graph depicting the number of neutrophils recovered during BAL in either VEGF+ or VEGF− mice who were either treated with vehicle control or Poly (I:C). FIG. 4E is a graph depicting the number of macrophages recovered during BAL in either VEGF+ or VEGF− mice who were either treated with vehicle control or Poly(I:C).

FIG. 5, comprising FIG. 5A through FIG. 5D, is a series of images depicting photomicrographs depicting the accumulation of inflammatory cells in lung tissue. FIG. 5A is a photomicrograph of lung tissue obtained from a VEGF− mouse treated with PBS. FIG. 5B is a photomicrograph of a VEGF+ mouse treated with PBS. FIG. 5C is a photomicrograph depicting a VEGF− mouse treated with Poly(I:C). FIG. 5D is a photomicrograph of a VEGF+ mouse treated with Poly(I:C).

FIG. 6, comprising FIG. 6A through FIG. 6D, is a series of images depicting photomicrographs depicting d-PAS stains in lung tissue. FIG. 6A is a photomicrograph of lung tissue obtained from a VEGF− mouse treated with PBS. FIG. 6B is a photomicrograph of a VEGF+ mouse treated with PBS. FIG. 6C is a photomicrograph depicting a VEGF− mouse treated with Poly(I:C). FIG. 6D is a photomicrograph of a VEGF+ mouse treated with Poly(I:C).

FIG. 7, comprising FIG. 7A and FIG. 7B, is a series of graphs depicting the effects of delaying Poly(I:C) administration on vascular responses. FIG. 7A is a graph depicting the effect of delaying Poly(I:C) administration for two days after Dox administration. FIG. 7B is a graph depicting the effect of delaying Poly(I:C) administration for two weeks after Dox administration.

FIG. 8, comprising FIG. 8A and FIG. 8B, is a series of graphs depicting the effects of delaying Poly(I:C) administration on the total number of inflammatory cells recovered during BAL. FIG. 8A is a graph depicting the effect of delaying Poly(I:C) administration for two days after Dox administration. FIG. 8B is a graph depicting the effect of delaying Poly(I:C) administration for two weeks after Dox administration.

FIG. 9, comprising FIG. 9A through FIG. 9C, is a series of images depicting the ability of Poly(I:C) to regulate VEGF-induced nitric oxise synthase (NOS) iNOS, eNOS, and protein kinase B (AKT). FIG. 9A is a pair of graphs depicting the effect of Poly(I:C) on VEGF-induced eNOS mRNA (left panel) and VEGF-induced iNOS mRNA (right panel). FIG. 9B is a photograph of a gel obtained from a Western blot depicting the ability of Poly(I:C) to regulate VEGF-induced eNOS protein (top gel). β-actin is used as a control (bottom gel). FIG. 9C is a photograph of a gel obtained from a Western blot depicting the effect of Poly(I:C) on VEGF-induced AKT activation. In the top gel, antibodies specific for Akt were used. In the middle gel, antibodies specific for phosphorylated AKT (p-AKT) were used. In the bottom gel, antibodies to actin were used as a control.

FIG. 10, comprising FIG. 10A and FIG. 10B, is a series of images depicting the ability of Poly(I:C) to reduce the inflammatory response in mice sensitized and challenged with ovalbumin (OVA). FIG. 10A is a pair of photomicrographs of inflammatory cells recovered from BAL fluids in OVA challenged mice treated with either PBS or Poly(I:C). FIG. 10B is a graph depicting the number of total and individual inflammatory cells recovered in BAL fluids from PBS-treated non-challenged mice (PBS-NC), Poly(I:C)-treated non-challenged mice (PoIyI:C-NC), PBS-treated OVA-challenged mice (PBS-OVA), and Poly(I:C) treated OVA challenged mice (Poly(I:C)-OVA). Abbreviations: Neu=neutrophilds, Lym=lymphocytes, Eos=eosinophils, Mac=macrophages.

FIG. 11 is a graph depicting the concentration of IL-13 in BAL fluids recovered from unchallenged- and OVA-challenged mice treated with either PBS or Poly(I:C).

FIG. 12, comprising FIG. 12A through FIG. 12D, is a series of images of photomicrographs depicting the effect of PBS or Gardiquimod (GDQM) on normal and VEGF-induced angiogenesis in mouse lung. FIG. 12A is a photomicrograph depicting the effect of PBS on angiogenesis in VEGF−mice. FIG. 12B is a photomicrograph depicting the effect of PBS on angiogenesis in VEGF+mice. FIG. 12C is a photomicrograph depicting the effect of GDQM on angiogenesis in VEGF−mice. FIG. 12D is a photomicrograph depicting the effect of GDQM on angiogenesis in VEGF+mice.

FIG. 13, comprising FIG. 13A and FIG. 13B, is a series of images depicting the effect of GDQM on VEGF-induced inflammation and hemorrhage. FIG. 13A is a graph depicting the effect of PBS or GDQM on the total number of inflammatory cells recovered in BAL fluids obtained from VEGF+ or VEGF−mice. FIG. 13B is a photograph depicting the effect of PBS or GDQM on hemorrhage in BAL fluids obtained from VEGF+ or VEGF−mice.

FIG. 14, comprising FIG. 14A through FIG. 14D, is a series of images depicting the effect of GDQM on VEGF-induced inflammation in mouse lung. FIG. 14A is a photomicrograph depicting the effect of PBS on VEGF−mice. FIG. 14B is a photomicrograph depicting the effect of PBS on VEGF+mice. FIG. 14C is a photomicrograph depicting the effect of GDQM on VEGF−mice. FIG. 14D is a photomicrograph depicting the effect of GDQM on VEGF+mice.

FIG. 15, comprising FIG. 15A through FIG. 15D, is a series of images depicting the effect of GDQM on VEGF-induced mucus metaplasia in mouse lung. FIG. 15A is a photomicrograph depicting the effect of PBS on VEGF−mice. FIG. 15B is a photomicrograph depicting the effect of PBS on VEGF+mice. FIG. 15C is a photomicrograph depicting the effect of GDQM on VEGF−mice. FIG. 15D is a photomicrograph depicting the effect of GDQM on VEGF+mice.

### DETAILED DESCRIPTION OF THE INVENTION

The present invention is related to the discovery that Toll-like receptor (TLR) agonists regulate VEGF-induced tissue responses including angiogenesis, vascular permeability, hemorrhage, inflammation, and mucus metaplasia. The present invention encompasses compositions and compounds comprising TLR agonists and methods of their use for treating, attenuating, alleviating, or preventing VEGF induced tissue responses which contribute to the clinical presentation in a number of disease states and conditions including, but not limited to, asthma, chronic obstructive pulmonary disease (COPD), acute lung injury (ALI), acute respiratory distress syndrome (ARDS), obstructive sleep apnea (OSA), idiopathic pulmonary fibrosis (IPF), tuberculosis, pulmonary hypertension, pleural effusion, and lung cancer.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “agonist,” as used herein, is a molecule that binds to a specific receptor and triggers a response. An agonist may be a naturally occurring molecule or it may be a synthetic molecule. An agonist may be an endogenous molecule, synthesized and present in an organism, or it may be an exogenous molecule, such as a drug, synthesized outside of an organism and administered to an organism, provided that the synthetic or exogenous agonist mimics the activity of the endogenous agonist. Agonists may be defined both in terms of their affinity and maximum efficacy for their cognate receptors.

The affinity of an agonist for its receptor is defined by its dissociation constant, i.e. how tightly a particular agonist binds to its receptor. Agonist-receptor affinities are influenced by non-covalent intermolecular interactions between the two molecules such as hydrogen bonding, electrostatic interactions, hydrophobic and Van der Waals forces.

The efficacy of an agonist refers to the ability of an agonist to induce a biological response in its target, for example a cell.

A full agonist binds to (i.e., has affinity for) and activates a receptor, displaying full efficacy for a receptor. A partial agonist also binds to and activates a receptor, but only has partial efficacy for a receptor as compared to a full agonist. A co-agonist works with other co-agonists to produce the desired effect together. An antagonist blocks a receptor from activation by agonists.

The phrase “at risk” as used herein refers to a subject with a greater than average likelihood of developing a disease or disorder associated with clinical features due to a VEGF-induced tissue response.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” or “attenuated” if the severity of a symptom of the disease, or disorder, the frequency with which such a symptom is experienced by a patient, or both, are reduced.

The term “dysregulation” as used herein is used describes an over- or under-expression of VEGF present and detected in a body sample obtained from an individual as compared to VEGF present in a sample obtained from one or more normal, not-at-risk individuals, or from the same individual at a different time point. In some instances, the level of VEGF expression is compared with an average value obtained from more than one not-at-risk individuals. In other instances, the level of VEGF expression is compared with a VEGF level assessed in a sample obtained from one normal, not-at-risk sample. In yet another instance, the level of VEGF expression in the putative at-risk individual is compared with the level of VEGF expression in a sample obtained from the same individual at a different time.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein “endogenous” refers to any material from or produced inside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in an inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “pulmonary leak” or “pulmonary permeability” as used herein refers to a VEGF-induced tissue response wherein the overexperssion of VEGF stimulates the growth of friable blood vessels that are prone to bleed. Pulmonary leak or pulmonary permeability is measured by the amount of blood present in BAL fluids collected from a subject, or by Evans Blue dye extravasation from blood vessels.

The term “recombinant DNA” as used herein is defined as DNA produced by joining pieces of DNA from different sources.

The term “recombinant polypeptide” as used herein is defined as a polypeptide produced by using recombinant DNA methods.

By the term “specifically binds,” as used herein, is meant a molecule, such as an antibody, which recognizes and binds to another molecule or feature, but does not substantially recognize or bind other molecules or features in a sample.

### Description

The present invention is related to the discovery that a TLR agonist can regulate a VEGF-induced tissue response including angiogenesis, vascular permeability, hemorrhage, inflammation, and mucus metaplasia. The present invention encompasses compositions and compounds comprising a TLR agonist for treating, alleviating, or preventing the physiological effects of a VEGF-induced tissue response.

The methods of the present invention comprise administering a composition or compound comprising a TLR agonist to a subject exhibiting a VEGF-induced tissue response or determined to be at risk for developing a VEGF-induced-induced tissue response. The methods of the present invention further comprise administering a composition or compound comprising a TLR agonist to a subject who has been diagnosed with asthma, COPD, ALI, ARDS, OSA, IPF, tuberculosis, pulmonary hypertension, pleural effusion, or lung cancer, or who has symptoms or signs of a VEGF-induced tissue response.

The invention may be practiced in any subject diagnosed with, or at risk of developing a VEGF-induced tissue response. VEGF-induced tissue responses are associated with many diseases and disorders. The subject may be diagnosed with or be at risk for developing asthma, COPD, ALI, ARDS, OSA, IPF, tuberculosis, pulmonary hypertension, pleural effusion, or lung cancer. Preferably, the subject is a mammal and more preferably, a human.

### I. Compositions: Toll-Like Receptor Agonists

Toll-like receptors (TLRs) are a class of pattern recognition receptors (PRR) comprising single membrane-spanning non-catalytic receptors that recognize molecules that are broadly shared by pathogens but distinguishable from host molecules, collectively referred to as pathogen-associated molecular patterns (PAMPs). They form a receptor superfamily with the Interleukin-1 receptors (Interleukin-1 Receptor/Toll-Like Receptor Superfamily) that all have in common a so called TIR (Toll-IL-1 receptor) domain. Most mammalian species have between ten and fifteen types of Toll-like receptors. TLR1 to TLR10 have been identified in humans.

When activated by an agonist binding to a TLR, TLRs recruit adapter protein molecules within the cytoplasm of cells in order to propagate a signal. Four adapter protein molecules are known to be involved in TLR signaling: MyD88, Tirap (also called Mal), Trif, and Tram. The adapters activate other molecules within the cell, including certain protein kinases (IRAK1, IRAK4, TBK1, and IICKi) that amplify the signal, and ultimately lead to the induction or suppression of genes that orchestrate the inflammatory response.

Naturally-occurring Toll-like receptor agonists can be molecules associated with microbial threats to an organism (i.e. pathogen or cell stress) and are highly specific to these threats (i.e. cannot be mistaken for self molecules). Examples of well-conserved features in pathogens that act as TLR agonists include bacterial cell-surface lipopolysaccharides (LPS), lipoproteins, lipopeptides and lipoarabinomannan; proteins such as flagellin from bacterial flagella; double-stranded RNA of viruses or the unmethylated CpG islands of bacterial and viral DNA, and certain other RNA and DNA (Table I). TLR agonists may also be synthetic molecules, provided that they specifically bind a TLR and regulate a VEGF-induced tissue response.

TLR agonists useful in the invention are well known in the art (Table I). TLR ligands include: triacy lipoproteins (TLR1 agonists); lipoproteins, gram positive peptidoglycans, lipoteichoic acids, fungi and viral glycoproteins (TLR2 agonists); a viral RNA mimetic comprising a synthetic double-stranded RNA analog known as polyinosinic-polycytidylic acid (poly(I:C)) (TLR3 agonists; Alexopoulou et al., 2001, Nature 413:732-738); lipopolysaccharides, viral glycoproteins, mycobacterial glycolipid lipoarrabinomannan (LAM), bacterial lipoproteins, peptidoglycans, zymosan (Akira et al., 2001, Nature Immunol 2:675-680; Aderem and Ulevitch, 2000, Nature 406:782-787) and Enterobacterial LPS (TLR4 agonists); flagellin (TLR5 agonists); diacyl lipoproteins (TLR6 agonists); small synthetic compounds such as the antiviral imidazoquinoline resiquimod (R-848; Hemmi et al., 2002, Nature Immunol. 3:196-200) and single-stranded RNA (TLR7 and TLR8 agonists); and unmethylated CpG DNA (TLR9 agonists; Hemmi et al., 2000, Nature 408:740-745; Bauer et al., 2001, Proc. Natl. Acad, Sci. U.S.A. 98:9237-9242). CpG motifs are non-methylated C-G dinucleotides flanked by two 5′ purines and three 3′ pyramidines. In mammalian genomic DNA these sequences are rare, and are generally methylated.

TABLE 1
Toll-like Receptors and their known Agonists

| Receptor | Known Ligand(s) | Known Adaptor(s) | Location |
|---|---|---|---|
| TLR1 | triacyl lipoproteins | MyD88/MAL | cell surface |
| TLR2 | lipoproteins; gram | MyD88/MAL | cell surface |
| | positive | | |
| | peptidoglycan; | | |
| | lipoteichoic acids; | | |
| | fungi; viral | | |
| | glycoproteins | | |
| TLR3 | double-stranded | TRIF | cell compartment |
| | RNA (as found in | | |
| | certain viruses), | | |
| | poly I:C | | |
| TLR4 | lipopolysaccharide; | MyD88/MAL/ | cell surface |
| | viral glycoproteins | TRIF/TRAM | |
| TLR5 | flagellin | MyD88 | cell surface |
| TLR6 | diacyl lipoproteins | MyD88.MAL | cell surface |
| TLR7 | small synthetic | MyD88 | cell compartment |
| | compounds; | | |
| | single-stranded | | |
| | RNA | | |
| TLR8 | small synthetic | MyD88 | cell compartment |
| | compounds; | | |
| | single-stranded | | |
| | RNA | | |
| TLR9 | unmethylated CpG | MyD88 | cell compartment |
| | DNA | | |
| TLR10 | unknown | unknown | cell surface |

Because TLRs are pattern recognition receptors and the structure of their agonists are well known, the invention shall not be construed to be limited to those agonists recited herein, but should be construed to encompass any compound, small molecule, peptide, or nucleic acid that specifically binds to a TLR and is able to modulate a VEGF-induced tissue response. New TLR agonists may be discovered using standard screening techniques well-known in the art. Test compounds for use in such screening methods can be small molecules, peptides, nucleic acids, or other drugs.

Known TLR are commercially available. In addition, TLR agonists may be obtained using standard methods known to the skilled artisan. Such methods include chemical organic synthesis or biological means. Biological means include purification from a biological source, recombinant synthesis and in vitro translation systems, using methods well known in the art.

### Peptides

When the TLR agonist is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N— and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a TLR agonist in accordance with the invention, a peptide is purified to remove contaminants. Any one of a number of a conventional purification procedures may be used to attain the required level of purity including, for example, reversed-phase high-pressure liquid chromatography (HPLC) using an alkylated silica column such as C₄—,C₈— or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### Nucleic Acids

When the TLR agonist comprises a nucleic acid, any number of procedures may be used for the generation of an isolated nucleic acid encoding the agonist as well as derivative or variant forms of the isolated nucleic acid, using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones of TLR agonists, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al.

An isolated nucleic acid of the present invention can be produced using conventional nucleic acid synthesis or by recombinant nucleic acid methods known in the art and described elsewhere herein (Sambrook et al., 2001, Molecular Cloning: A Laboratory Manual, Cold Spring Harbor Laboratory Press, New York) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang et al., (1979, Meth. Enzymol., 68: 90) and U.S. Pat. No. 4,356,270. The phosphotriester method can be used in the present invention to synthesize an isolated TLR agonist nucleic acid.

In addition, the compositions of the present, invention can be synthesized in whole or in part, or an isolated TLR agonist nucleic acid can be conjugated to another nucleic acid using organic synthesis such as the phosphodiester method, which has been used to prepare a tRNA gene. See Brown et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

Known TLR agonists include small synthetic compounds such as polyinosinic:polycytidylic acid (poly I:C), a synthetic double-stranded RNA as well as synthetic single stranded RNA. Thus, oligonucleotide agents are incorporated herein and include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (1994, Nucleic Acids Res. 22: 2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body.

As nucleic acids are polymers of subunits or monomers, many of the modifications described below occur at a position which is repeated within a nucleic acid, e.g., a modification of a base, or a phosphate moiety, or a non-linking O of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. A component can be attached at the 3′ end, the 5′ end, or at an internal position, or at a combination of these positions. For example, the component can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking O position may only occur at one or both termini, or may only occur in a terminal region, e.g., at a position on a terminal nucleotide or in the last 2, 3, 4, 5, or 10 nucleotides of the oligonucleotide. The 5′ end can be phosphorylated.

For increased nuclease resistance and/or binding affinity to the target, an oligonucleotide agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, O-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

Preferred substitutents include but are not limited to 2′-methoxyethyl, 2′-OCH3, 2′-O-allyl, 2′-C- allyl, and 2′-fluoro.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diary) amino, heteroaryl amino,or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

One way to increase resistance is to identify cleavage sites and modify such sites to inhibit cleavage. For example, the dinucleotides 5′-UA-3′, 5′-UG-3′, 5′-CA-3′, 5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. In certain embodiments, all the pyrimidines of the miRNA inhibitor carry a 2′-modification, and the miRNA inhibitor therefore has enhanced resistance to endonucleases.

In addition, to increase nuclease resistance, the 2′ modifications can be used in combination with one or more phosphate linker modifications (e.g., phosphorothioate). The so-called “chimeric” oligonucleotides are those that contain two or more different modifications.

With respect to phosphorothioate linkages that serve to increase protection against RNase activity, the miRNA inhibitor can include a phosphorothioate at at least the first, second, or third internucleotide linkage at the 5′ or 3′ end of the nucleotide sequence. In one embodiment, the miRNA inhibitor includes a 2′-modified nucleotide, e.g., a 2′-deoxy, 2′-deoxy-2′-fluoro, 2′-0-methyl, 2′-O-methoxyethyl (2-O-MOE), 2′-O- aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In a preferred embodiment, the miRNA inhibitor includes at least one 2′-O-methyl-modified nucleotide, and in some embodiments, all of the nucleotides of the miRNA inhibitor include a 2′-O-methyl modification.

The 5′ -terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

The oligonucleotide can be constructed using chemical synthesis and/or enzymatic ligation reactions using procedures known in the art. For example, an oligonucleotide can be chemically synthesized using naturally occurring nucleotides or variously modified nucleotides designed to increase the biological stability of the molecules or to increase the physical stability of the duplex formed between the oligonucleotide and target nucleic acids, e.g., phosphorothioate derivatives and acridine substituted nucleotides can be used. Other appropriate nucleic acid modifications are described herein. Alternatively, the oligonucleotide can be produced biologically using an expression vector into which a nucleic acid has been subcloned in an antisense orientation (i.e., RNA transcribed from the inserted nucleic acid will be of an antisense orientation to a target nucleic acid of interest (e.g., an mRNA, pre-mRNA, or an miRNA).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

### TLR Agonists Comprising Molecules Present in Pathogen Cell Wall

Bacterial and fungal cell walls contain a number of molecules long known to evoke an immune response and stimulate cytokine release, including but not limited to lipopolysaccharides, peptidoglycans, lipoteichoic acid, and glycoproteins. These cell wall molecules may be obtained by using recombinant techniques, where appropriate, as described above, or by extracting the molecules from the cell walls of various pathogens using techniques well-known in the art (Fuller, 1938, Brit. J. Exp. Pathol. 19:130-9; Matted, 1948, Lancet 2:255-6; Salton et al, 1951, Biochim. Biophys. Acta 7:177-97).

All bacteria containing a cell wall, and particularly Gram positive bacteria, possess a specific component of the cell wall called peptidoglycan. The peptidoglycan provides structural support to the bacterial cell. Peptidoglycan is made up of alternating sugar units (N-acetylglucosamine and N-acetylmuramic acid). The sugars are joined by short peptide chains that consist of four amino acids. The sugars and tetrapeptides are crosslinked by a simple peptide bond.

When the TLR is a peptidoglycans, a peptidoglycan extract may be produced by extracting the bacteria (and peptidoglycan) by heating in water and acid. The crude extract is subsequently centrifuged and/or filtered to remove insoluble components. The extract can be further purified using ultrafiltration or other suitable methods for removing the reagents, salts, and other impurities prior to lyophilization to produce the final dry powder which may be reconstituted using any suitable vehicle.

When the TLR agonist is a lipopolysaccharide, a LPS may be obtained from a washed and dried bacterial mass by a modified phenol/water extraction using methods well-known in the art (O. Westphal et al., 1965, Bacterial Lipopolysaccharides, Extraction with Phenol-Water and Further Applications of the Procedure, Meth. Carbohydr. Chem., 5:83-91; Inzana, 1983, J. Infect. Dis. 148:492-499; Johnson et al., 1976, Can. J. Microbiol. 22:29-34; Kurt-Jones et al., 2004, J. Endotoxin Res. 10:419-424). The extract can be further purified using ultrafiltration or other suitable methods for removing the reagents, salts, and other impurities prior to lyophilization to produce the final dry powder which may be reconstituted using any suitable vehicle.

### Small Molecules

When the TLR agonist is a small molecule, a small molecule agonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### Molecules for Use as Test Compounds

A test compound useful in the present invention is a potential TLR agonist and may be a peptide, a nucleic acid, a small molecule, or other drug that specifically binds to a TLR, thereby regulating a VEGF-induced tissue-response. Test molecules may be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145).

Examples of methods for the synthesis of molecular libraries may be found in the art, for example, in: DeWitt et al., 1993, Proc. Natl. Acad. Sci. USA 90:6909-6913; Erb et al., 1994, Proc. Natl. Acad. Sci. USA 91:11422-11426; Zuckermann et al., 1994, J. Med. Chem. 37:2678-2685; Cho et al., 1992, Science 261:1303-1305; Carell et al., 1994, Angew. Chem. Int. Ed. Engl. 33:2059-2061; Carell et al., 1994, Angew. Chem. Int. Ed. Engl. 33:2061-2064; and Gallop et al., 1994, J. Med. Chem. 37:1233-1251.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869), or phage (Scott and Smith, 1990, Science 249:386-390; Devlin, 1990, Science 249:404-406; Cwirla et al:, 1990, Proc. Natl. Acad. Sci. USA 87:6378-6382; and Felici, 1991, J Mol. Biol. 222:301-310).

The resulting libraries of candidate molecules may be screened to determine their efficacy as TLR agonists or as a regulator of a VEGF-induced tissue response using any technique well known in the art. Such techniques include, but are not limited to, high-throughput bioassays, such as binding assays or activity based assays, to determine a molecule's ability to specifically bind to or activate a TLR; structural analysis such as X-ray crystallography; drug fragment-based analysis, including binding assays; computational analysis (e.g. Target Infomatics Platform, Eidogen; Passadena, Calif.); animal-based, tissue-based, or cell-based assays, to determine a molecule's effect on a VEGF-induced tissue response.

### II. Methods

The invention provides a method of regulating a VEGF-induced tissue response in a mammal. In another embodiment, the present invention provides a method of treating a mammal diagnosed with a disease or disorder wherein VEGF expression is dysregulated. In another embodiment, the invention further provides a method of treating a mammal diagnosed with a disease or disorder wherein VEGF-induced tissue response is a component of the disease or disorder. In still another embodiment, the present invention encompasses methods of treating pulmonary inflammation, angiogenesis, vascular permeability or leak, hemorrhage, and mucus metaplasia. In yet another embodiment, the invention further comprises a method of treating pulmonary diseases and disorders where VEGF-induced tissue responses are a component of the disease or disorder. A non-limiting example of a disease or disorder that may be treated using the methods of the invention, includes, but is not limited to acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In another embodiment, the present invention further comprises a method of decreasing VEGF-induced activation of the AKT-eNOS/iNOS pathway. In yet another embodiment, the present invention provides a method of preventing the development of a VEGF-induced tissue response in a subject at-risk for developing a VEGF-induced tissue response.

The methods of the invention comprise administering a therapeutically effective amount of at least one TLR agonist to a mammal wherein a TLR agonist attenuates a VEGF-induced tissue response including but not limited to vascular permeability, hemorrhage, angiogenesis, inflammation, edema, effusion, and tissue remodeling. In another embodiment, the methods of the invention comprise administering a therapeutically effective amount of at least one TLR agonist to a mammal wherein a TLR agonist attenuates dysregulation of VEGF expression. In another embodiment, the methods of the invention comprise administering a therapeutically effective amount of at least one TLR agonist to a mammal wherein a TLR agonist is used to treat a mammal diagnosed with a disease or disorder wherein VEGF-induced tissue response is a component of the disease or disorder. In still another embodiment, the methods of the invention comprise administering a therapeutically effective amount of at least one TLR agonist to a mammal wherein a TLR agonist is used to treat pulmonary inflammation, pathological angiogenesis, vascular permeability, hemorrhage, and mucus metaplasia. In yet another embodiment, the methods of the invention comprise administering a therapeutically effective amount of at least one TLR agonist to a mammal wherein a TLR agonist is used to treat pulmonary diseases and disorders where VEGF-induced tissue responses are a component of the disease or disorder. A non limiting example of a disease or disorder that may be treated using the methods of the invention, includes, but is not limited to, acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In another embodiment, the methods of the invention comprise administering a therapeutically effective amount of at least one TLR agonist to a mammal wherein a TLR agonist attenuates VEGF-induced activation of the AKT-eNOS/iNOS pathway.

The subject may be diagnosed with a disease or disorder wherein the disease or disorder has a VEGF-induced tissue response as part of the disease's clinical features. In another embodiment, the subject may be at-risk of developing a disease or disorder wherein the disease or disorder has a VEGF-induced tissue response as part of the disease's clinical features. Examples of a disease or disorder which may be treated using the methods of the present invention include but are not limited to acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer. In a preferred embodiment the subject is a mammal. In a more preferred embodiment he subject is a human.

Methods of prophylaxis (i.e., prevention or decreased risk of disease), as well as reduction in the frequency or severity of symptoms associated with VEGF-induced tissue responses or any related disease or disorder, are encompassed by the present invention.

The method of the invention comprises administering a therapeutically effective amount, of at least one TLR agonist to a mammal wherein a TLR agonist is used either alone or in combination with other therapeutic agents to treat a subject. A TLR agonist may be administered either, before, during, after, or throughout the administration of said therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, brondhodilaters, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

Regulating VEGF activity and VEGF-induced tissue responses via activation of at least one Toll-like receptor can be accomplished using any method known to the skilled artisan. In one embodiment of the invention a TLR agonist specifically binds to a TLR and consequently alters expression of a gene. In another embodiment of the invention, a TLR agonist binds to a TLR receptor and regulates the expression of a downstream effector, such as at least one kinase, which in turn antagonizes VEGF activity. In another embodiment, a TLR agonist binds to a TLR and regulates nictric oxide synthase (NOS) activity. In still another embodiment of the invention, a TLR agonist binds to a TLR receptor and regulates the expression of a downstream effector which in turn augments VEGF activity. In yet another embodiment of the invention, a TLR agonist binds to a TLR and consequently activates of numerous cytokines and other endogenous signaling molecules, including but not limited to IL-β1, IL-6, IL-8, TNF-α, iNOS, IP-10, RANTES and MCP-C, any of which may augment or antagonize VEGF activity and VEGF-induced tissue response. A TLR agonist may be any type of compound, including but not limited to, a peptide, a nucleic acid, and a small molecule, or combinations thereof.

### Methods of Delivering a TLR Agonist to a Cell

The present invention comprises a method for regulating a VEGF-induced tissue response in a mammal, said method comprising administering a therapeutic amount TLR agonist to said mammal. In particular, the invention includes a method for attenuating a VEGF-induced tissue response such as angiogenesis, inflammation, vascular permeability, hemorrhage, and mucus metaplasia which are features of a number of diseases and disorders.

A TLR can be located on a cell surface or within a sub-cellular compartment. Therefor, isolated nucleic acid-based TLR agonists can be delivered to a cell in vitro or in vivo using viral vectors comprising one or more isolated TLR agonist sequences. Generally, the nucleic acid sequence has been incorporated into the genome of the viral vector. The viral vector comprising an isolated TLR agonist nucleic acid described herein can be contacted with a cell in vitro or in vivo and infection can occur. The cell can then be used experimentally to study, for example, the effect of an isolated TLR agonist in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated TLR agonist nucleic acid sequence, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated TLR agonist nucleic acid of the present invention. Ex vivo gene therapy has been described, for example, in Kasid et al., Proc. Natl. Acad. Sci. USA 87:473 (1990); Rosenberg et al, New Engl. J Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising an isolated TLR agonist nucleic acid can be implanted into a subject or experimental animal model for delivery or used in in vitro experimentation to study cellular events mediated by TLR activation.

Various viral vectors can be used to introduce an isolated TLR agonist nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated TLR agonist nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated TLR agonist nucleic acid can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Various forms of an isolated TLR agonist nucleic acid, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated TLR agonist nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell.

Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690). However, as demonstrated by the data disclosed herein, an isolated snRNA of the present invention is a stable nucleic acid, and thus, may not be susceptible to degradative enzymes. Further, despite the fact that the aqueous space of typical liposomes may be too small to accommodate large macromolecules, the isolated TLR agonist nucleic acid of the present invention is relatively small, and therefore, liposomes are a suitable delivery vehicle for the present invention. Methods of delivering a nucleic acid to a cell, tissue or organism, including liposome-mediated delivery, are known in the art and are described in, for example, Feigner (Gene Transfer and Expression Protocols Vol. 7, Murray, E. J. Ed., Humana Press, New Jersey, (1991)).

In other related aspects, the invention includes an isolated TLR agonist nucleic acid operably linked to a nucleic acid comprising a promoter/regulatory sequence such that the nucleic acid is preferably capable of delivering an isolated TLR agonist nucleic acid. Thus, the invention encompasses expression vectors and methods for the introduction of an isolated TLR agonist nucleic acid into or to cells.

Such delivery can be accomplished by generating a plasmid, viral, or other type of vector comprising an isolated TLR agonist nucleic acid operably linked to a promoter/regulatory sequence which serves to introduce the TLR agonist into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated TLR agonist nucleic acid may be accomplished by placing an isolated TLR agonist nucleic acid, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### Pharmaceutical Compositions and Therapies

Administration of a TLR agonist in a method of treatment can be achieved in a number of different ways, using methods known in the art. Such methods include, but are not limited to, providing exogenous TLR agonist to a subject, expressing a TLR agonist, or expressing a TLR agonist expression cassette.

In one embodiment, an exogenous TLR agonist is administered to a subject. The exogenous agonist may also be a peptide, a nucleic acid, a small molecule, or other drug, or a combination thereof. The TLR may also be a hybrid or fusion protein to facilitate, for instance, delivery to target cells or efficacy. In one embodiment, a hybrid protein may comprise a tissue-specific targeting sequence.

In another embodiment, an expression vector comprising an expression cassette encoding a TLR agonist is administered to a subject. An expression cassette may comprise a constitutive or inducible promoter. Such promoters are well known in the art, as are means for genetic modification. Expression vectors and methods for the introduction of exogenous DNA into cells with concomitant expression of the exogenous DNA in the cells are described, for example, in Sambrook et al. (2001, Molecular Cloning: A Laboratory Manual, Cold Spring Harbor Laboratory Press, Cold Spring Harbor, N.Y.), and in Ausubel et al. (eds, 2005, Current Protocols in Molecular Biology, John Wiley & Sons, New York, N.Y.). In one embodiment, a cell comprising an expression vector of the invention is administered to a subject. Thus, the invention encompasses a cell comprising an isolated nucleic acid encoding a TLR agonist or fusion protein of the invention.

Any expression vector compatible with the expression of a TLR agonist or fusion protein of the invention is suitable for use in the instant invention, and can be selected from the group consisting of a plasmid DNA, a viral vector, and a mammalian vector. The expression vector, or a vector that is co-introduced with the expression vector, can further comprise a marker gene. Marker genes are useful, for instance, to monitor transfection efficiencies. Marker genes include: genes for selectable markers, including but not limited to, G418, hygromycin, and methotrexate, and genes for detectable markers, including, but not limited to, luciferase and GFP. The expression vector can further comprise an integration signal sequence which facilitates integration of the isolated polynucleotide into the genome of a target cell.

The therapeutic and prophylactic methods of the invention thus encompass the use of pharmaceutical compositions comprising a TLR agonist, fusion protein or small molecule of the invention and/or an isolated nucleic acid encoding a TLR agonist, fusion protein or small molecule of the invention to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient comprising a TLR agonist, a pharmaceutical composition of the invention may further comprise one or more additional pharmaceutically active agents. The compositions and methods of the present invention can be used in combination with other treatment regimens, including virostatic and virotoxic agents, antibiotic agents, antifungal agents, anti-inflammatory agents, as well as combination therapies, and the like. The invention can also be used in combination with other treatment modalities, such as chemotherapy, cryotherapy, hyperthermia, radiation therapy, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising at least one TLR agonist of the invention and an instructional material which describes, for instance, administering a TLR agonist to a subject as a prophylactic or therapeutic treatment as described elsewhere herein. In an embodiment, this kit further comprises a (preferably sterile) pharmaceutically acceptable carrier suitable for dissolving or suspending the therapeutic composition, comprising a TLR agonist of the invention, for instance, prior to administering the molecule to a subject. Optionally, the kit comprises an applicator for administering the inhibitor. In one embodiment of the invention, the applicator is designed for pulmonary administration of the TLR agonist.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Generation of Inducible Transgenic Mice

The CC10-rtTA-VEGF₁₆₅ transgenic mice used in this study were generated and used as described previously (Lee et al., 2004, Nature Medicine 10:1095-103), which reference is incorporated herein in its entirety. These animals were housed under barrier conditions in the animal facility at Yale University School of Medicine. In these experiments 6-8 week old transgenic (Tg+) animals and their transgene negative littermate controls (Tg−) were randomized to receive water with doxycycline (dox) (0.5 mg/ml) or normal water. Two weeks later they were sacrificed and the VEGF-induced phenotype was evaluated as described below (Lee et al., 2004, Nature Medicine 10:1095-103).

Treatment with Agonists of Innate Immune Pathways

Poly(I).Poly(C) double strand RNA (Poly(I:C)), an activator of toll-like receptor-3 (TLR3) and other innate immunity pathways, was purchased from Amersham Biosciences (Piscataway, N.J., USA). Lipopolysaccharide (LPS) an activator of TLR4 was obtained from Sigma-Aldrich (St. Louis, Mo.) and Gardiquimod (GDQM) a TLR7 ligand was obtained from Invivogen, (San Diego, Calif., USA).

To define the effects of these agents on the tissue responses induced by VEGF, two experimental procedures were used. In the first, a pretreatment protocol was used. In these experiments the noted innate immunity agonist (30 mg/per mouse unless otherwise indicated) or its vehicle control (PBS) were administered intranasally and aspirated into the lungs of randomized Tg+ and Tg− mice starting one day before the Dox water treatment. The doses were administered to the mice every other day during the 2 week doxyxycline (dox) treatment interval.

To determine if the innate immunity agonists altered established VEGF-induced tissue responses, a “post treatment” protocol was used in selected experiments. In these experiments the innate immunity agonist was administered 2 days or 14 days after the doxycycline and VEGF-induced responses were evaluated 2 weeks later.

### Characterization of VEGF-Induced Tissue Response

Bronchoalveolar Lavage (BAL) and Histologic Analysis: BAL was performed and BAL cellular evaluations were undertaken as previously described (Lee et al., 2004, Nature Medicine 10:1095-103; Zhu et al., 1999, J. Clin. Invest. 103:779-88). Histologic evaluations including hematoxyln and eosin (H&E) and diastase-periodic acid schiff (d-PAS) stains were performed in the Research Histology Laboratory at Yale. Pulmonary hemorrhage was evaluated by quantitating BAL red blood cell number with a Coulter counter (Beckman Coulter).
Evans Blue Dye Extravasation: Evans Blue dye was purchased from Sigma-Aldrich (Cat#E2129), and was dissolved in 0.9% saline at a final concentration of 5 mg/ml prior to use. The permeability alterations of murine lungs were assessed as previously described (Lee et al., 2004, Nature Medicine 10:1095-103).
BAL Total Protein Measurement: Bronchoalveolar lavage (BAL) was collected as previously reported. A Dc Protein Assay Kit from Bio-Rad Laboratories (Hercules, Calif.) was used to measure BAL total protein levels according to manufacturer's instructions. The optical density of each sample was read at 750 nm wavelength with a Smartspec 3000 Spectrophotometer (Bio-Rad Laboratories).
Whole Lung dry/wet Ratios: Animal lungs were excised en bloc after intravascular perfusion and weighed. They were then put into a SpeedVac system (ThermoSavant; Milford, Mass.), desiccated at 60° C. overnight, and reweighed.
CD31 Immunohistochemistry (IHC): Rat anti monoclonal antibody against CD31 (BD Pharmingen, San Jose, Calif.) was applied to the explanted tracheas to detect mouse tracheal vascular endothelial cells. These evaluations were performed as described^{7,27,28}

### Western Blot Evaluations:

These experiments used antibodies specific for Akt or phosphorylated (p) Akt (Danvers Mass.), FAK or pFAK (BioSource, International; Camarillo Calif.), P13K or pP3K (Cell Signalling), eNOS and iNOS (Santa Cruz BioTechnology, Santa Cruz Calif., USA) and MUC-5AC (NeoMarkers; Fremont). Western blot analysis was performed using standard protocols and visualized using the ECL system (Amersham Biosciences; Piscataway, N.J.).

### ELISA Evaluations:

ELISA assay kits for VEGF and IL-13 were purchased from R&D Systems (Minneapolis, Minn.).

### RNA Isolation and Quantitative Real-time PCR:

Total RNA was isolated from fresh mouse lung with TRIzol reagent (Invitrogen; Carlsbad, Calif.) following the manufacturer's instructions. Quantification of mouse lung mRNA was performed by quantitative real-time PCR using ABI 7500 fast real-time PCR system (Applied Biosystems; Foster City, Calif.) and power SYBR green PCR master mix kit with appropriately specific primers (Applied Biosystems; Foster City, Calif.).

### Statistics

Student's t test was used to evaluate statistical significance between experimental groups and controls. Data are expressed as means ±SEMs. All experiments were repeated at least three times. A P-value≦0.05 was considered statistically different.

The results of the experiments presented in the Examples are now described.

TABLE 2
Effect of TLR agonist administration on
VEGF-induced tissue responses.

| | | | | Total cells |
|---|---|---|---|---|
| Treatment | TLR | Hemorrhage | Vascularity (%) | (×10⁴) |
| PBS | Control | +++++ | 43 | 50 |
| Poly(I:C) | TLR3 | + | 28.7 | 22 |
| Gardiquimod | TLR7 | ++ | 32.4 | 29 |
| CPG | TLR9 | +++ | 42.1 | 39 |
| LPS | TLR4 | +++++ | 32.2 | 156 |

### Example 1

### Poly(I:C) Regulation of VEGF-Induced Vascular Responses

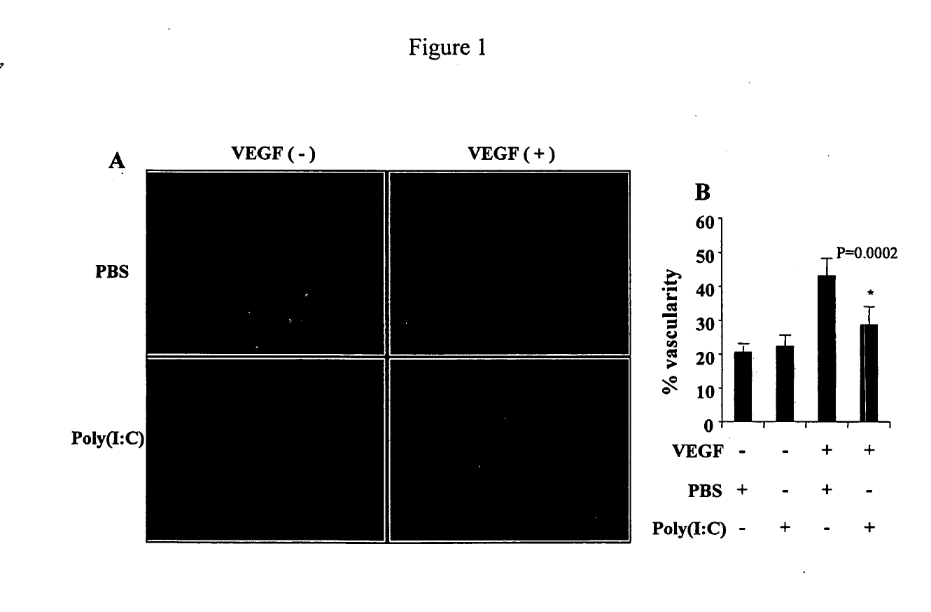

To begin to define the effects of TLR-agonists on VEGF-induced responses, Tg+ and Tg− mice were treated with poly(I:C) starting 1 day before the administration of Dox. The ability of transgenic VEGF to induce angiogenesis, vascular leak, and pulmonary hemorrhage was then evaluated. FIG. 1A and FIG. 1B demonstrate that this treatment markedly decreased the ability of VEGF to induce tissue angiogenesis. This can be seen in the CD31-like immunoreactivity in the FIG. 1A and the morphometric quantification of the percentage of the surface area of the airway that was covered by blood vessels (FIG. 1B).

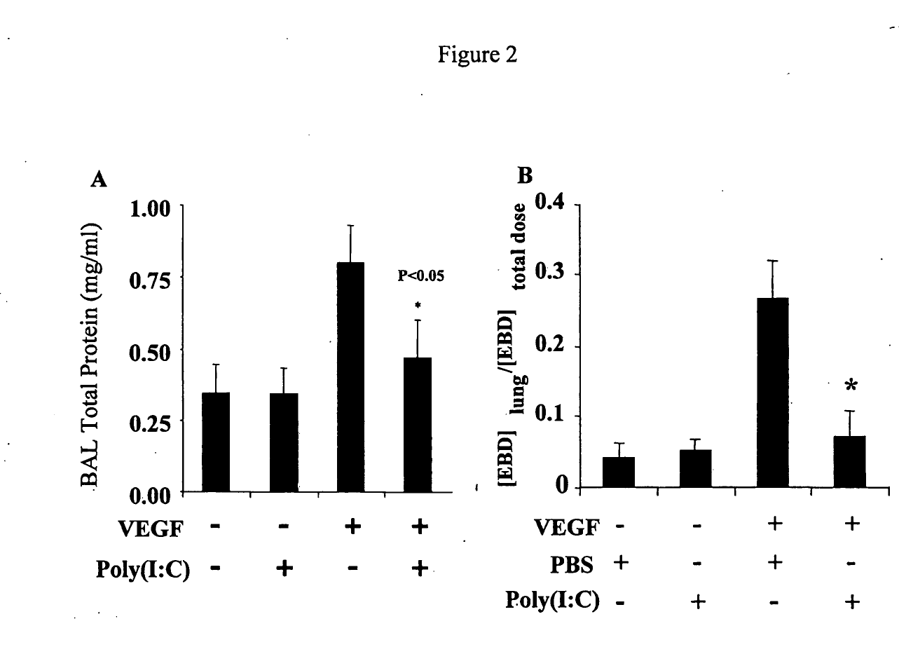

Poly(I:C) decreases vascular permeability. FIG. 2A demonstrates that there are lower levels of protein in BAL fluids from Tg+ mice treated with poly(I:C) when compared to Tg+ mice treated with vehicle. FIG. 2B demonstrates that there are lower levels of Evan's blue dye leakage in lungs from Tg+ mice treated with poly(I:C) when compared to Tg+ mice treated with the control vehicle.

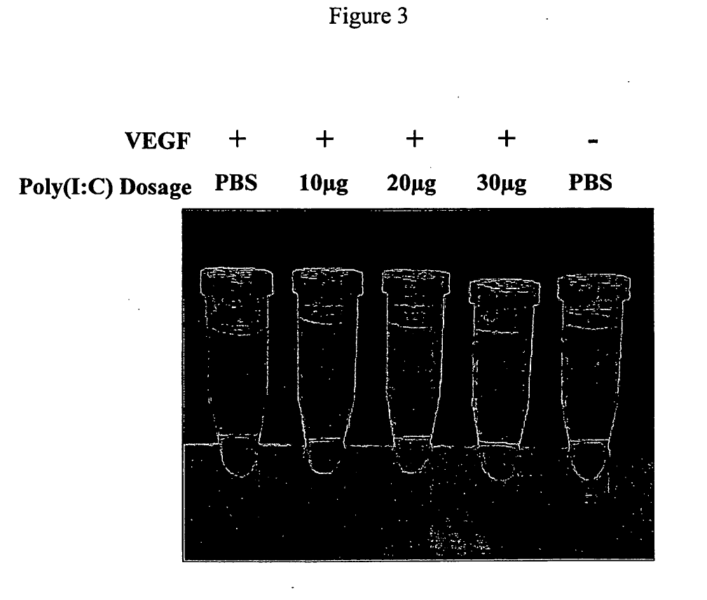

In the present model system, and others, the overproduction of VEGF stimulates the growth of friable blood vessels that are prone to bleed. This can be seen in the red (bloody) BAL fluids obtained from these Tg+ mice (FIG. 3). As can be seen in FIG. 3, this bleeding was significantly decreased in Tg+ mice treated with poly(I:C).

### Example 2

### Poly(I:C) Regulation of VEGF-Induced Extra-Vascular Responses

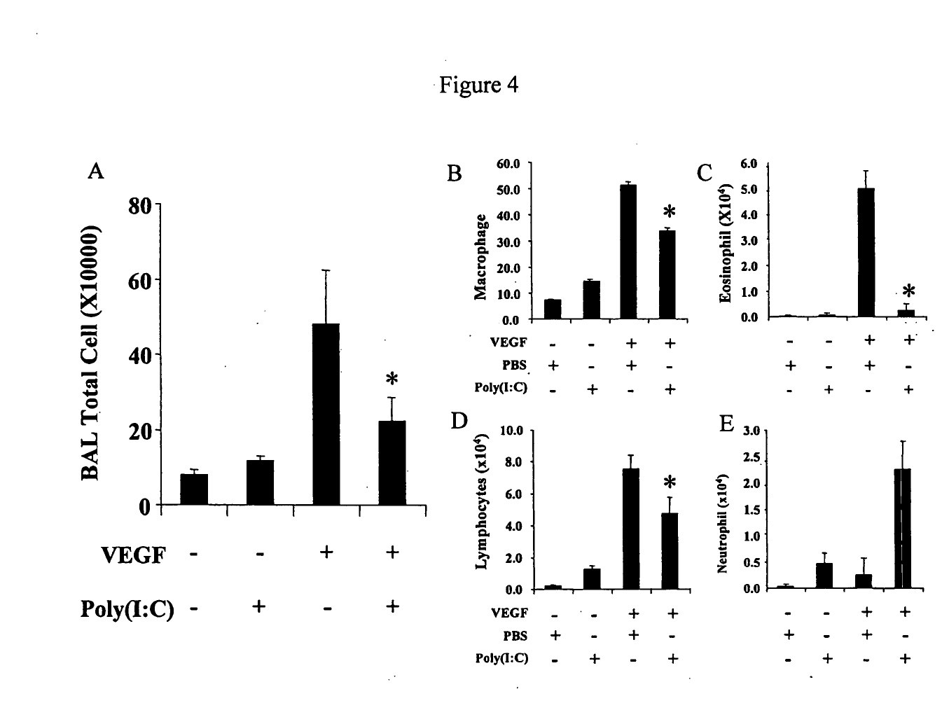

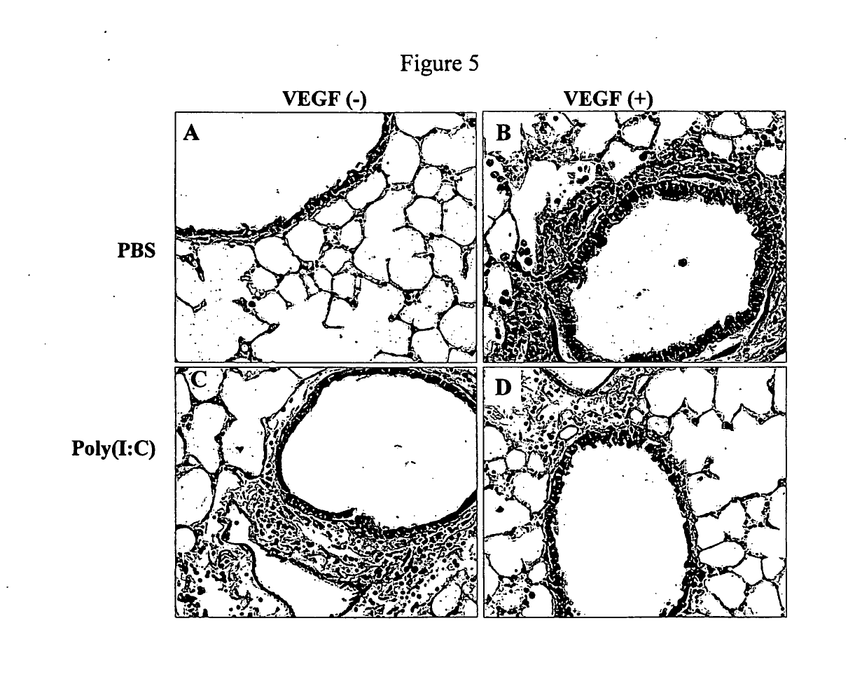

To begin to define the effects of TLR-agonists on extra-vascular VEGF responses Tg+ and Tg− mice were treated with Poly(I:C) starting 1 day before the administration of Dox. The ability of transgenic VEGF to induce tissue inflammation and mucus metaplasia were then evaluated. FIGS. 4(A-E) demonstrates that this treatment markedly decreased the total recovery of inflammatory cells during BAL. It also demonstrates that the recovery of macrophages, eosinophils and lymphocytes were all significantly decreased (*p<0.05). FIGS. 5(A-D) demonstrates that similar alterations in inflammatory cell accumulation were seen in lung tissues. Specifically, modest levels of inflammation were seen in VEGF Tg+ mice that received vehicle and Tg− mice that received only poly(I:C). In contrast, lower levels of inflammation were seen in Tg+ that were treated with Poly(I:C).

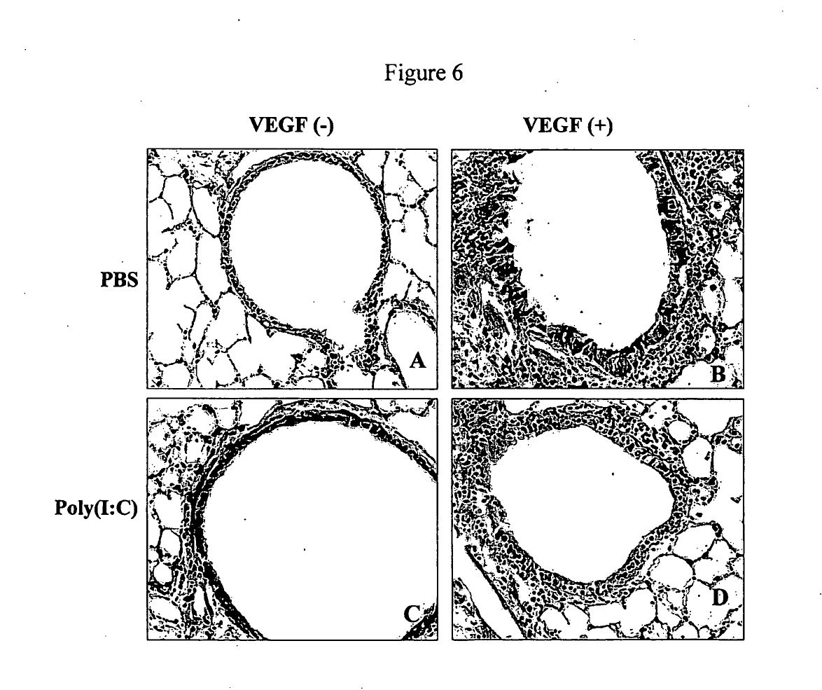

Mucus metaplasia with goblet cell hyperplasia is well described in human airways diseases such as asthma. As can be seen in the d-PAS stains in FIG. 6, transgenic VEGF induced a similar response in the murine lung. Interestingly, this response was significantly decreased by treatment with poly(I:C) (FIG. 6D). VEGF also stimulated mucus secretion and this secretion was decreased by Poly(I:C) (data not shown).

### Example 3

### Effects of Poly(I:C) on BAL VEGF

The inhibitory effects that were noted above could be due to the ability of Poly(I:C) to inhibit VEGF effector responses or its ability to decrease the production of transgenic VEGF. To differentiate amongst these options we measured the levels of VEGF in BAL fluids from Tg+ mice treated with Poly(I:C) or control vehicle. These studies demonstrated that Poly(LC) treatment did not alter the levels of production of transgenic VEGF and instead appeared to alter the ability of VEGF to induce tissue effector responses.

### Example 4

### Poly(LC) Treatment is Effective in Ongoing Disease

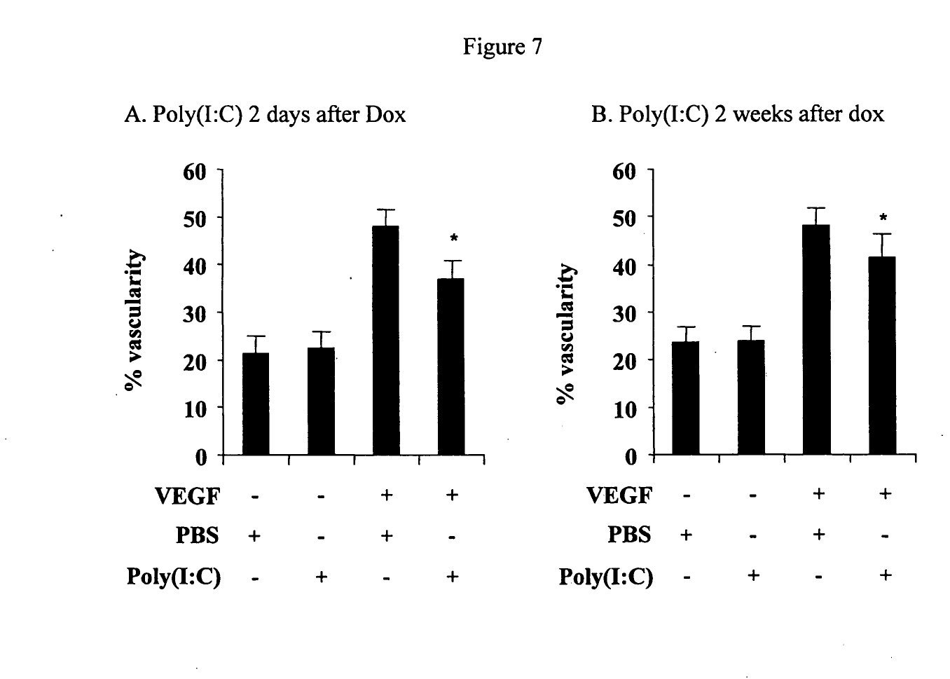

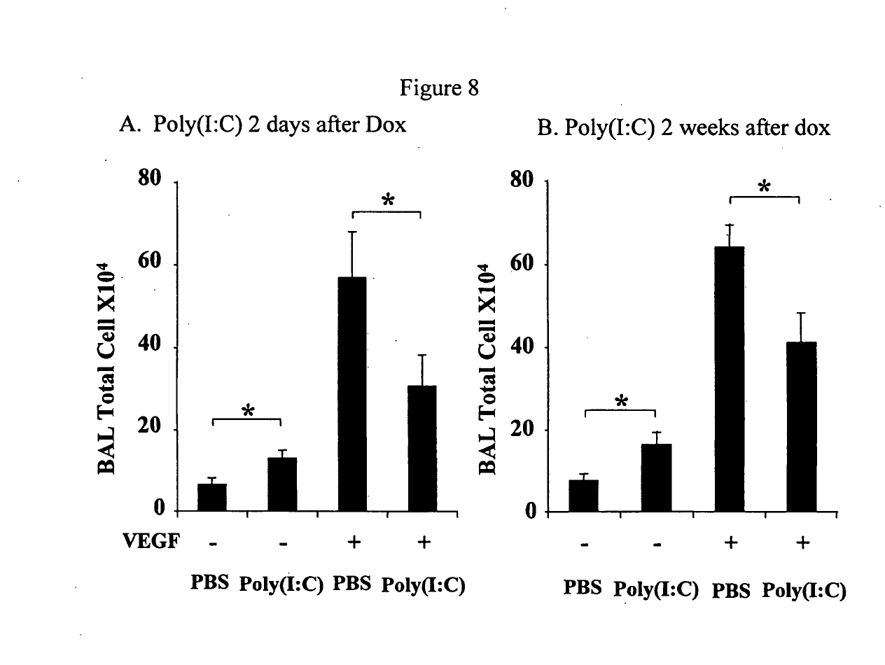

Physicians frequently need to treat patients with established diseases. To see if treatment with Poly(I:C) could alter angiogenesis and or inflammation in mice with ongoing VEGF-induced alterations we compared the effects of Poly(I:C) in Tg+ mice when it was administered 24 hours before and 2 days and 14 days after the VEGF transgene was activated with dox. As shown in FIGS. 7 and 8, Poly(I:C) caused a significant decrease in vascular responses and inflammation at both time points.

### Example 5

### Poly(I:C) Regulation of Protein Kinase B (AKT) and Nitric Oxide Synthase (NOS)

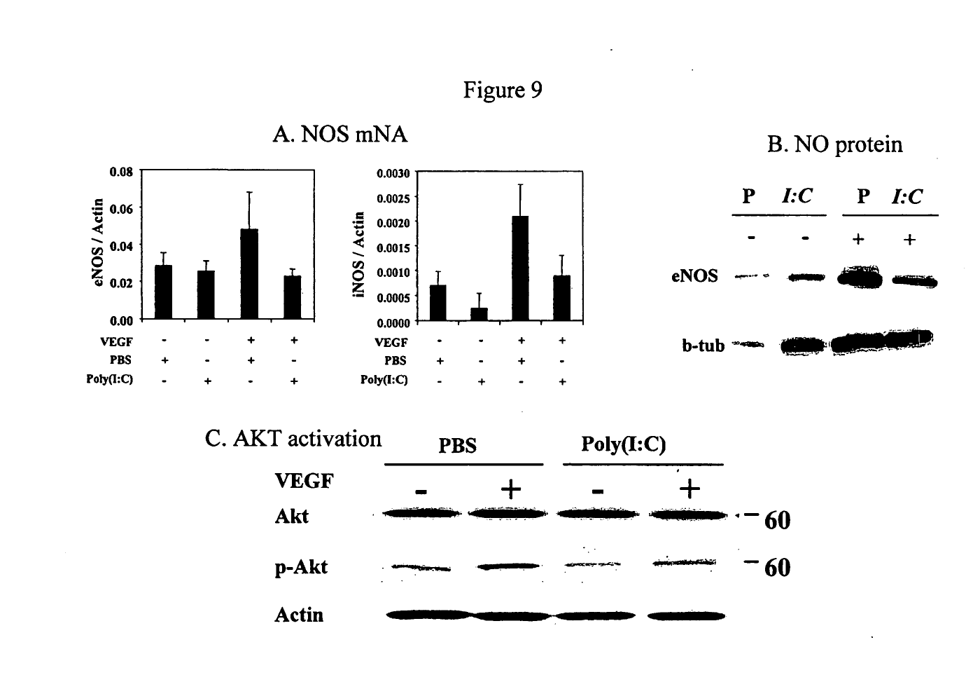

Previous studies from demonstrated that VEGF induces its tissue responses in the lung, at least in part, via its ability to stimulate eNOS and iNOS. In addition, AKT is known to regulate NOS in a variety of tissues. Thus, studies were undertaken to determine if Poly(I:C) altered the ability of VEGF to activate AKT and stimulate eNOS and or iNOS. As shown in FIG. 9, transgenic VEGF was a potent stimulator of eNOS and iNOS mRNA accumulation and AKT phosphorylation/activation. As can also be seen in FIG. 9, Poly(I:C) treatment caused a significant decrease in each of these responses (*p<0.05). This suggests that Poly(I:C) may mediate its effects, at least in part, via its ability to decrease the ability of VEGF to activate the AKT-eNOS/iNOS pathway.

### Example 6

### Poly(I:C) Inhibits Asthma-Like Th2 Inflammation

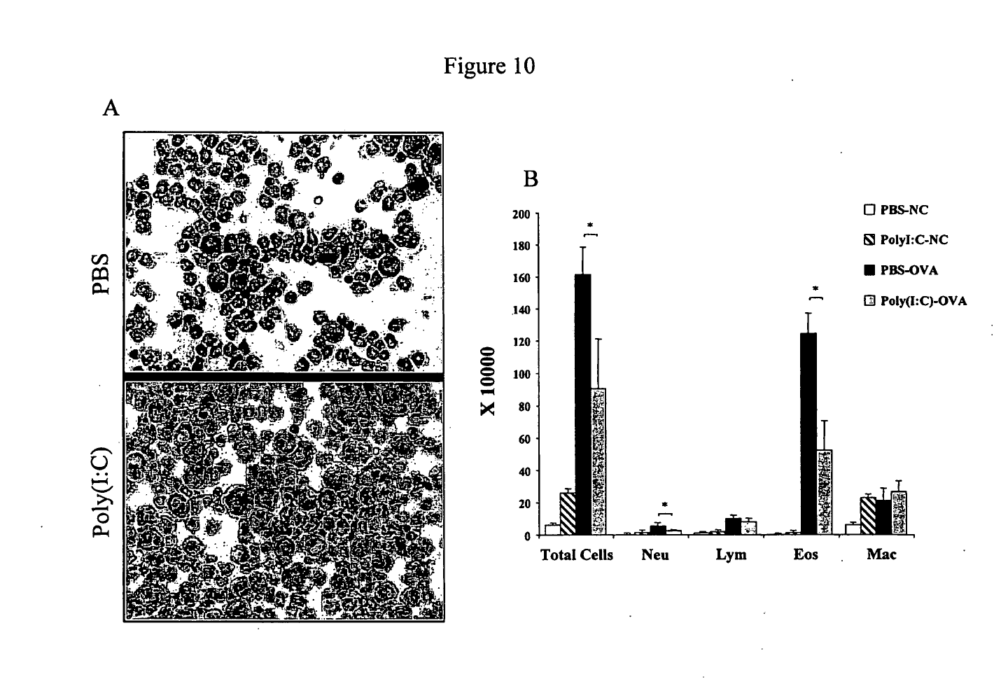

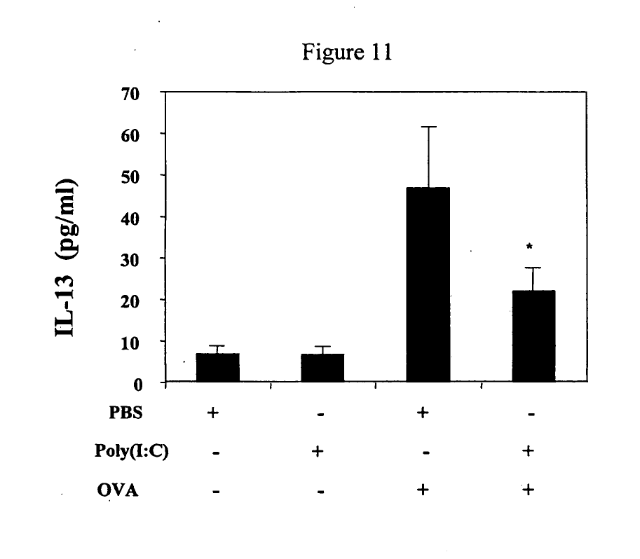

Previous studies demonstrated that VEGF is required for antigen-induced Th2 inflammation and IL-4 and IL-13 elaboration (Lee et al., 2004, Nature Medicine 10:1095-103). Thus, Poly(I:C) might be able to decrease Th2 inflammation via its ability to alter VEGF effector responses. To test this hypothesis the standard ovalbumin (OVA) sensitization and challenge lung Th2 inflammation model was used. In this model, mice are sensitized to OVA in the presence of alum and then receive aerosol OVA challenges. As can be seen in FIGS. 10 and 11, this causes a brisk eosinophil-rich inflammatory response (FIG. 10) and a significant increase in IL-13 production (FIG. 11). Poly(I:C) treatment caused a significant decrease in these inflammatory and cytokine responses. This highlights the ability of IIA to regulate Th2 inflammation.

### Example 7

### Specificity of the Poly(I:C) Effect

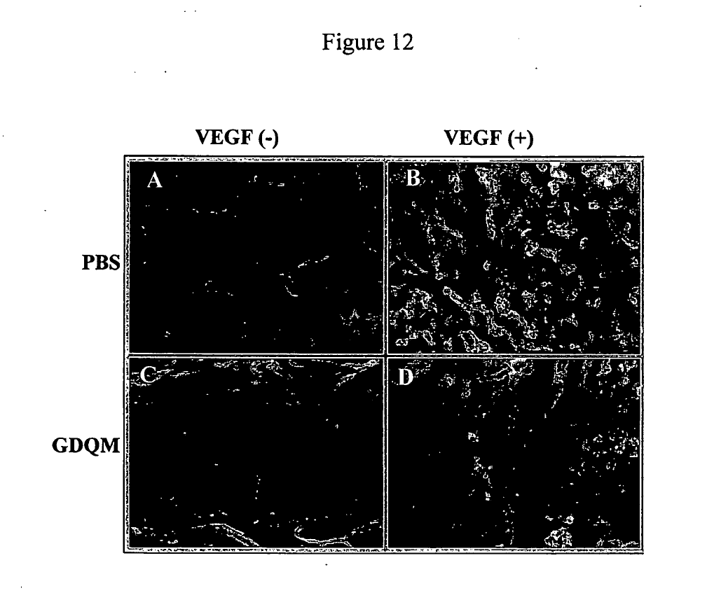

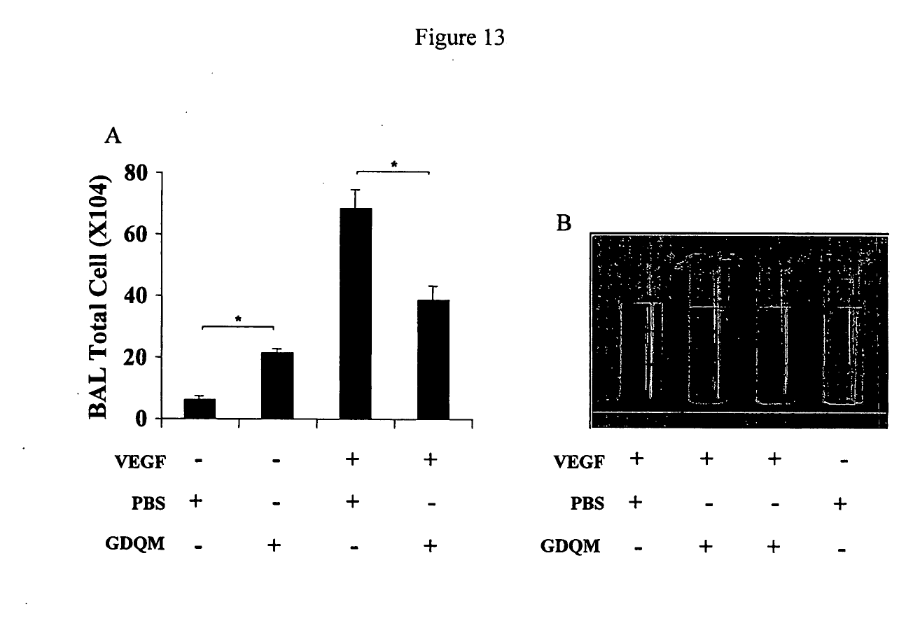

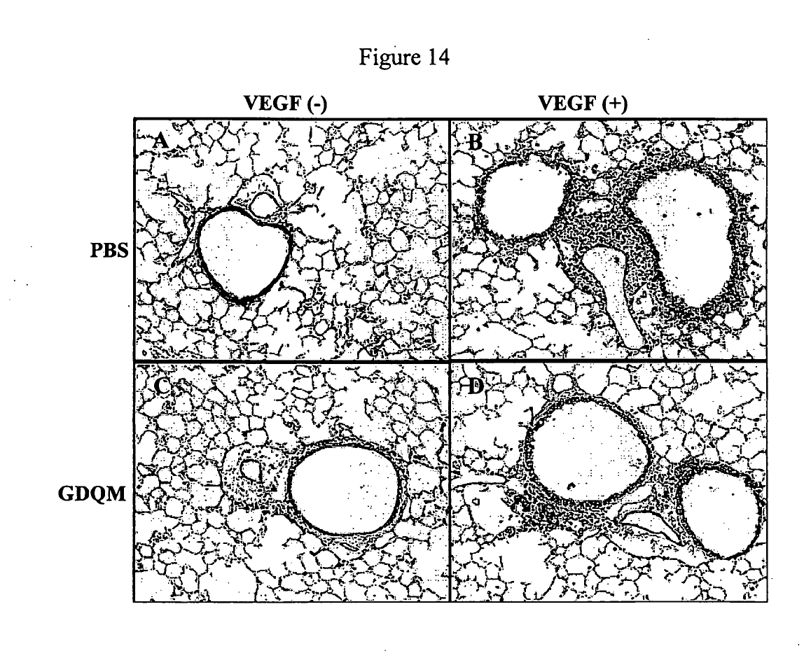

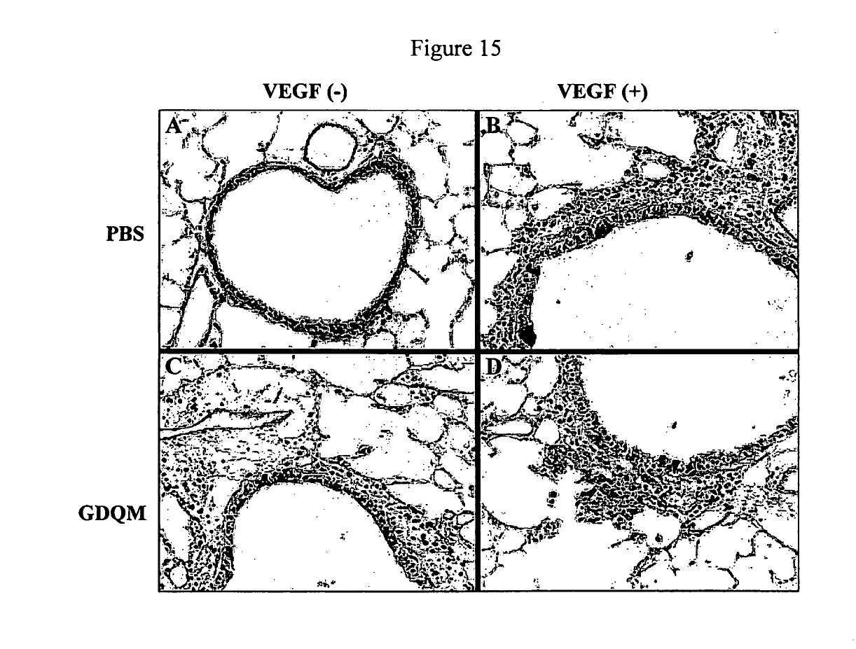

Studies were next undertaken to determine if other TLR-agonists altered VEGF-induced tissue responses in a manner that was similar to Poly(I:C). As can be seen in FIGS. 12-15, the TLR7 ligand GDQM decreased VEGF-induced angiogenesis, inflammation, hemorrhage, and mucus metaplasia. Similar effects were not seen with the vehicle controls.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of regulating a vascular endothelial growth factor (VEGF)-induced tissue response in a mammal, said method comprising administering to said mammal a therapeutically effective amount of at least one toll-like receptor (TLR) agonist, and further wherein when said TLR agonist is administered to said mammal, said VEGF-induced tissue response is regulated in said mammal.

2. The method of claim 1, wherein said mammal is a human.

3. The method of claim 1, wherein said VEGF-induced tissue response comprises increased angiogenesis, tissue inflammation, vascular permeability, vascular leak, hemorrhage, or mucus metaplasia.

4. The method of claim 1, wherein said mammal has been diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer.

5. The method of claim 1, wherein said TLR agonist is administered in combination with at least one other therapeutic agent.

6. The method of claim 5, wherein said TLR agonist is administered before, during, or after said therapeutic agent, or a combination thereof.

7. The method of claim 5, wherein said therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof.

8. The method of claim 1, where a TLR is administered in combination with other treatment modalities, such as chemotherapy, cryotherapy, hyperthermia, radiation therapy, or a combination thereof.

9. The method of claim 1, wherein said TLR agonist specifically binds to a TLR3, a TLR7, a TLR9, a TLR4, or a combination thereof.

10. The method of claim 1, wherein said TLR agonist comprises a poly(I:C), Gardiquimod, a CpG, a LPS, or a combination thereof.

11. A method of treating a vascular endothelial growth factor (VEGF)-induced tissue response in a mammal, said method comprising administering to said mammal a therapeutically effective amount of at least one toll-like receptor (TLR) agonist, and further wherein when said TLR agonist is administered to said mammal, said VEGF-induced tissue response is attenuated in said mammal.

12. The method of claim 11, wherein said mammal is a human.

13. The method of claim 11, wherein said VEGF-induced tissue response comprises increased angiogenesis, tissue inflammation, vascular permeability, vascular leak, hemorrhage, or mucus metaplasia.

14. The method of claim 11, wherein said mammal has been diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer.

15. The method of claim 11, wherein said TLR agonist is administered in combination with at least one other therapeutic agent.

16. The method of claim 15, wherein said TLR agonist is administered before, during, or after said therapeutic agent, or a combination thereof.

17. The method of claim 15, wherein said therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof.

18. The method of claim 11, where a TLR is administered in combination with other treatment modalities, such as chemotherapy, cryotherapy, hyperthermia, radiation therapy, or a combination thereof.

19. The method of claim 11, wherein said TLR agonist specifically binds to a TLR3, a TLR7, a TLR9, a TLR4, or a combination thereof.

20. The method of claim 11, wherein said TLR agonist comprises a poly(I:C), Gardiquimod, a COG, a LPS, or a combination thereof.

21. A method of preventing a vascular endothelial growth factor (VEGF)-induced tissue response in a mammal, said method comprising administering to said mammal a therapeutically effective amount of at least one toll-like receptor (TLR) agonist, and further wherein when said TLR agonist is administered to said mammal, said VEGF-induced tissue response is prevented in said mammal.

22. The method of claim 21, wherein said mammal is a human.

23. The method of claim 21, wherein said VEGF-induced tissue response comprises increased angiogenesis, tissue inflammation, vascular permeability, vascular leak, hemorrhage, or mucus metaplasia.

24. The method of claim 21, wherein said mammal is at risk of developing at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer.

25. The method of claim 21, wherein said TLR agonist is administered in combination with at least one other therapeutic agent.

26. The method of claim 25, wherein said TLR agonist is administered before, during, or after said therapeutic agent, or a combination thereof.

27. The method of claim 25, wherein said therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof.

28. The method of claim 21, where a TLR is administered in combination with other treatment modalities, such as chemotherapy, cryotherapy, hyperthermia, radiation therapy, or a combination thereof.

29. The method of claim 21, wherein said TLR agonist specifically binds to a TLR3, a TLR7, a TLR9, a TLR4, or a combination thereof.

30. The method of claim 21, wherein said TLR agonist comprises a poly(l:C), Gardiquimod, a CpG, a LPS, or any combination thereof.

31. The method of treating a mammal diagnosed with at least one disease or disorder selected from the group consisting of: acute lung injury (ALI), acute respiratory distress syndrome (ARDS), asthma, chronic obstructive pulmonary disease (COPD), obstructive sleep apnea (OSA), idiopathic pulmonary fibrosis (IPF), tuberculosis, pulmonary hypertension, pleural effusion, and lung cancer, said method comprising administering to said mammal a therapeutically effective amount of at least one toll-like receptor (TLR) agonist.

32. The method of claim 31, wherein said TLR agonist is administered in combination with at least one other therapeutic agent.

33. The method of claim 32, wherein said TLR agonist is administered before, during, or after said therapeutic agent, or a combination thereof.

34. The method of claim 32, wherein said therapeutic agent is selected from the list consisting of a virostatic agent, a virotoxic agent, an antibiotic, an antifungal agent, an anti-inflammatory agent, an antidepressant, an anxiolytic, a pain management agent, a steroid, an antihistamine, an antitussive, a muscle relaxant, a bronchodilator, a beta-agonist, an anticholinergi, a mast cell stabilizer, a leukotriene modifier, a methylxanthine, or a combination thereof.

35. The method of claim 31, where a TLR is administered in combination with other treatment modalities, such as chemotherapy, cryotherapy, hyperthermia, radiation therapy, or a combination thereof.

36. The method of claim 31, wherein said TLR agonist specifically binds to a TLR3, a TLR7, a TLR9, a TLR4, or a combination thereof.

37. The method of claim 31, wherein said TLR agonist comprises a poly(I:C), Gardiquimod, a CpG, a LPS, or a combination thereof.

38. The method of claim 31, wherein said mammal is a human.
